(19) United States
(12) Patent Application Publication
(10) Pub. No.: US 2024/0399007 A1
(43) Pub. Date: Dec. 5, 2024

(54) DISINFECTION DEVICE FOR A MEDICAL HELMET

(71) Applicant: THI TOTAL HEALTHCARE INNOVATION S.R.L., Rovigo (IT)

(72) Inventors: Giorgio ROSATI, San Cesareo RM (IT); Giorgio GAINO, Borgo Veneto PD (IT); Lorenzo SECCO, Treviso TV (IT); Daniele FORMAGGIO, Este PD (IT)

(73) Assignee: THI TOTAL HEALTHCARE INNOVATION S.R.L., Rovigo (IT)

(21) Appl. No.: 18/698,934

(22) PCT Filed: Nov. 17, 2022
(86) PCT No.: PCT/IB2022/061074

§ 371 Date: Apr. 5, 2024

(30) Foreign Application Priority Data
Nov. 26, 2021 (IT) 102021000029966

(51) Int. Cl. A61L 2/26; A42B 3/00; A61L 2/22; A61L 2/24

(52) U.S. Cl. CPC A61L 2/26; A42B 3/006; A61L 2/22; A61L 2/24; A61L 2202/122; A61L 2202/14; A61L 2202/15; A61L 2202/16; A61L 2202/24

(57) ABSTRACT

An automatic disinfection device for surgical helmets is disclosed. The device includes a box-shaped disinfection chamber configured to house the helmet needing disinfection inside and presenting prevalent extension in a sagittal plane, means for supplying a disinfectant substance, in communication with the interior of said disinfection chamber and means of printing a label applicable to the helmet and containing information about the disinfection.

### TECHNICAL FIELD OF THE INVENTION

The present invention relates to a device configured to disinfect a helmet of the type used by a healthcare worker, in particular a surgeon, during an operation.

### BACKGROUND

Over the past two decades, systems for protecting the head and face of a healthcare worker based on a helmet, to be worn during surgery have become very common. Traditionally, such systems also include a hood, or cape, that covers the helmet and is equipped with a lens-a transparent visor shield-attached to the fabric of the hood. The latter is designed to fit over an open front frame of the helmet.

As mentioned, the system is generally worn by the healthcare worker during surgery, e.g. orthopaedic surgery, and offers superior performance to that of masks and common face shields because it covers the entire head of the healthcare worker, creating a sterile barrier between him and the patient. In addition, the helmet generally includes a ventilation system for forced air circulation within the environment of the surgeon's head. This air circulation counteracts sweating and helps to keep the surgeon cool and oxygenated, thereby increasing his comfort level.

In addition, the above systems also offer valuable protection for the patient against contamination from the surgeon and other healthcare workers, as they prevent hair, dandruff and saliva droplets from falling to the surgical field and thus the possibility of wound infection.

Examples of helmets of the above type are described, for example, in WO2017/120562A2 and WO2012/111030A1.

Known surgical helmets generally have a lattice structure made up of one or more struts and thus a relatively complex shape, including, as mentioned, ventilation components. For this reason, prodromal disinfection operations prior to donning can be time-consuming, complicated and in some cases unreliable.

### SUMMARY OF THE INVENTION

The technical problem posed and solved by the present invention is therefore primarily that of providing a disinfection device for a surgical helmet, which makes it possible to overcome the aforementioned drawbacks with reference to the known art.

This problem is solved by a device according to claim 1.

Preferred features of the present invention are the subject matter of the dependent claims.

In the context of the present description, by reference to known terminology, spatial attributions such as “sagittal”, “frontal”, “anterior”, “transverse”, “lateral”, “posterior”, “upper”, “lower” and derivatives refer to the position of the helmet in the worn configuration, in association with anatomical definitions of the body of the operator wearing the helmet.

The device of the invention defines a disinfection chamber, and preferably a pair of chambers, each suitable for receiving a surgical helmet. Each disinfection chamber has a closed box-like conformation, with a prevailing extension in a sagittal plane, and in particular in an antero-posterior direction and in a vertical direction, and a thickness in a transverse direction reduced with respect to the other two dimensions.

Each chamber is selectively accessible for the introduction of the respective helmet and then closable, for example by means of a hatch or window in one or more of its perimeter walls that define the internal compartment for the helmet.

Each chamber is in communication with means for selectively supplying a disinfectant substance, configured to feed a controlled quantity, or volume, of disinfectant into one or each chamber. Advantageously, the disinfectant is supplied in nebulised form. In an embodiment, the device may be configured, by means of a corresponding control unit, to sequentially perform a disinfection step, or sanitisation, and a cleaning step with a detergent.

Furthermore, the device may include means for printing a label, the latter containing, for example, a confirmation of disinfection, the date, the time, any parameters of the disinfection process and/or the substance(s) used and/or various operational information.

The label may also be sterile and is immediately applicable on the helmet once removed from the respective disinfection chamber.

Preferably, the device comprises a support base which may include said disinfectant supplying means and/or said control unit.

In an embodiment thereof, the device further includes a user interface for operating and controlling the disinfection, preferably arranged at the support base.

The device is thus specifically configured for disinfection of a surgical helmet, which is performed effectively, with limited space requirements and, advantageously, also in fully automatic mode.

Other advantages, features and methods of use of the present invention will be apparent from the following detailed description of its embodiments, presented for illustrative and non-limiting purposes.

### BRIEF DESCRIPTION OF FIGURES

Reference will be made to the figures of the accompanying drawings, in which:

FIGS. 1A, 1B and 1C show a schematic sagittal, top perspective and side perspective view, respectively, of a preferred embodiment of the disinfection device according to the present invention;

FIG. 1D shows a perspective view of the device of FIG. 1A, in an opening configuration allowing the insertion of a surgical helmet into a disinfection chamber of said device;

FIG. 2 shows a side perspective view of a device in a variant embodiment of that shown in FIG. 1A, when used for independent or combined disinfection of two helmets;

FIG. 3 shows an exemplary block diagram of the device components of FIG. 1A or FIG. 2.

### DETAILED DESCRIPTION OF PREFERRED EMBODIMENTS

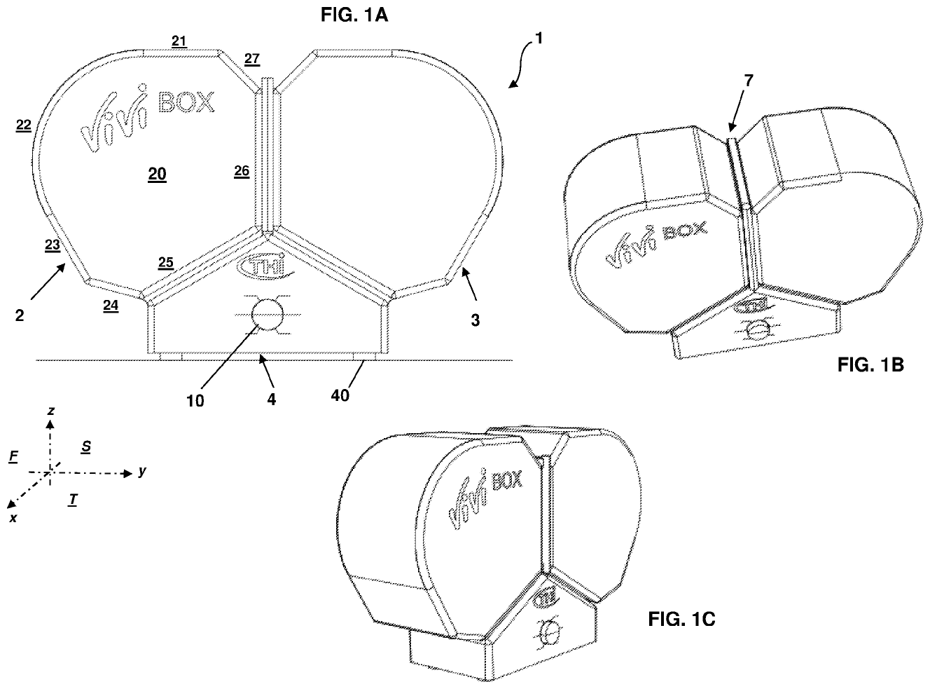

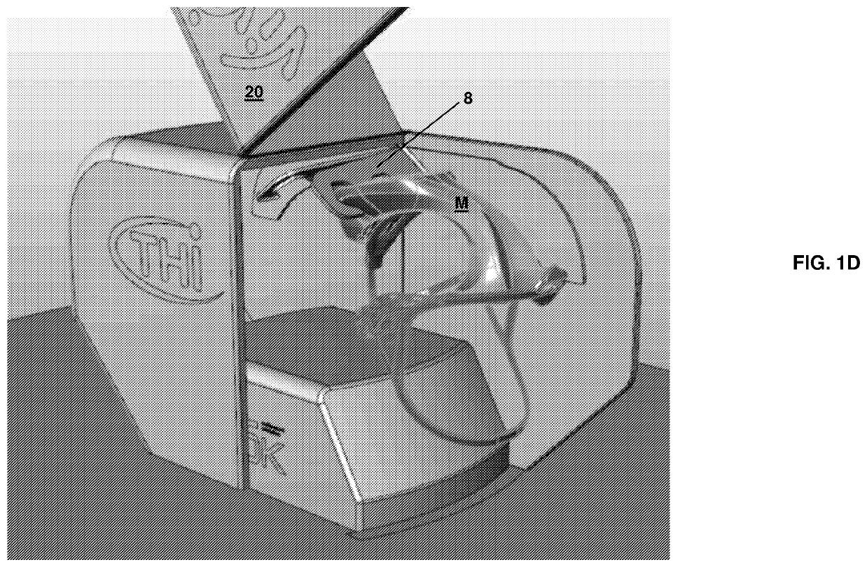

With reference to FIGS. 1A, 1B and 1C, a disinfection device for surgical helmets according to a preferred embodiment of the invention is collectively denoted 1.

For immediate reference, a Cartesian tern (x, y, z) defined according to the position each helmet assumes within the device 1 and according to the arrangement of the helmet itself corresponding to its configuration worn by a standing operator (anatomical position) is shown.

Thus, the xz plane (denoted by F) corresponds to an arrangement substantially parallel to a frontal plane of the operator, the xy plane (T) to an arrangement parallel to a transverse plane and the zy plane(S) to a sagittal plane.

The device 1 comprises a pair of disinfection chambers, 2 and 3 respectively, arranged juxtaposed parallel to the plane F and each configured to receive a respective surgical helmet.

Each of the disinfection chambers 2, 3 has a closed box shape, with prevalent extension according to the sagittal plane S, in particular according to the antero-posterior y direction and the vertical z direction.

The conformation of each of the chambers 2, 3 comprises opposing sagittal walls, one of which is visible in FIG. 1A and denoted 20.

In the embodiment of FIGS. 1A-1D, each sagittal wall 20 is delimited by a broken line profile connected towards the outside of the device 1. In particular, on said profile delimiting each sagittal wall 20 there is identified, starting from above, a substantially rectilinear antero-posterior section 21, a descending connecting section 22 having an arcuate profile, a first substantially rectilinear descending section 23, a second, also substantially rectilinear, descending section 24 less inclined than the first section 23, a substantially rectilinear ascending section 25, a vertical section 26 at the interface between the two chambers 2 and 3 and a further substantially rectilinear ascending section 27.

Overall, therefore, each chamber has an outwardly convex profile in the sagittal plane S.

The two chambers 2, 3 each have an independent internal compartment to receive their respective helmets. Between them, a structural septum 7 is defined with substantially vertical extension and parallel to the plane F.

Each of the chambers 2, 3 may present a closable hatch or opening for the introduction of the surgical helmet within it, or allow an opening movement obtained by a dislocation, for example by rotation, of one or more of the respective walls, as shown in FIG. 1D.

Preferably, each of the chambers 2, 3 has transparent peripheral walls or a transparent viewing window, in order to also allow a visual check of the presence of a helmet and/or a related disinfection operation. Below the two chambers 2, 3, at the inclined walls associated with the sections 25, a support base 4 is defined, for example provided with feet 40.

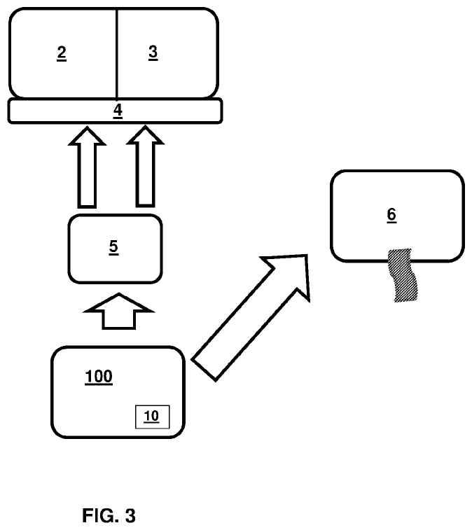

Referring also to the diagram in FIG. 3, within the base 4 can be housed operational components of the device 1, for example a control unit 100 and means 5 for supplying a disinfectant substance, in communication with the interior of the chambers 2 and 3. The control unit 100 may be associated with a user interface for controlling the device 1, for example a knob 10.

Disinfectant supplying means 5 are advantageously configured to feed a controlled amount, or volume, of disinfectant into one or each of the disinfection chambers 2, 3, preferably in nebulised form.

The device 1 further comprises, as exemplified only in FIG. 3, means 6 configured to generate and print a label applicable on the helmet, also advantageously arranged at base level 4 and controlled by the unit 100. The label may carry information about the disinfection having taken place, for example date, time, parameters of the disinfection process and/or of the substance(s) used or even an identifier of the device used.

The control unit 100 can be programmed to perform one or more disinfection or helmet cleaning operations, or cycles, so that the device 1 can operate automatically.

The mode of operation may involve first supplying a sanitiser and then a cleaning solution. Typically, as mentioned, the means for supplying will involve nebulization.

For example, a specific combination of ionic silver, citric acid and lactic acid can be used as a disinfectant solution, in addition to all products suitable for disinfection by nebulisation.

The control unit 100, moreover, may control signalling means arranged at the base 4, for example lights indicating the operational status of the device 1, a disinfection operation in progress and/or the presence of a helmet inside a chamber.

Equally, the signalling means may indicate the state of charge of the device 1, if a self-power supply is also provided, or the need for maintenance.

The device is preferably equipped with suitable sensors to detect the correct closure of the chambers and any deviations from the execution of the disinfection operation programmed by the control unit 100.

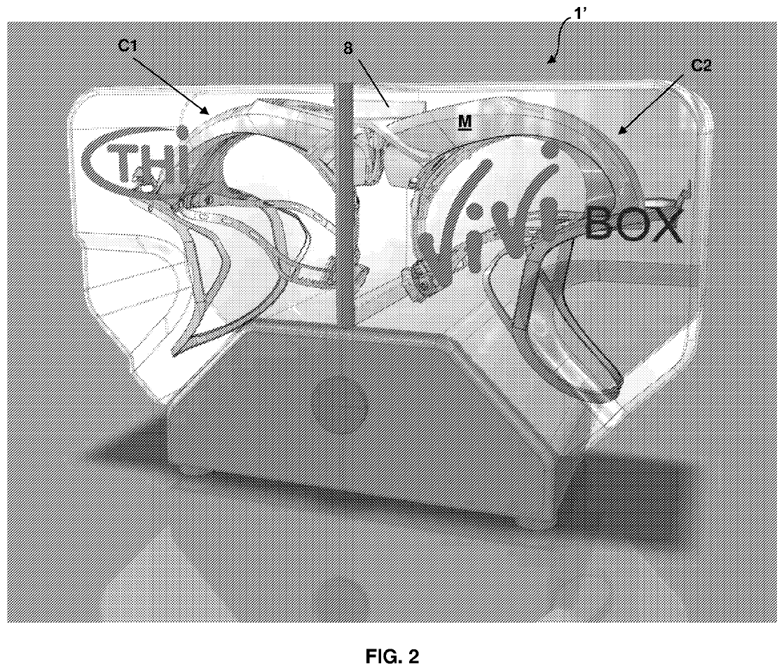

FIG. 2 shows two surgical helmets C1 and C2 each housed in a respective chamber of an embodiment variant of the device of the invention, denoted herein by 1′. The device of FIG. 2 has disinfection chambers with a more squarish/polyhedral conformation than the device of FIG. 1A.

Overall, the helmet C1 or C2 can be made as described in WO2017/120562A2.

FIGS. 1D and 2 also show means 8 of retaining each helmet. In the example shown, the means 8 comprise a snap-on coupling system acting bilaterally at a rear part of a central post M of the helmet itself.

According to the described configuration, the device 1 is easily transportable and in particular portable.

The present invention has so far been described with reference to preferred embodiments thereof. It is to be understood that there may be other forms of embodiment pertaining to the same inventive core, as defined by the scope of protection of the claims below.

1. A portable automatic disinfection device for one or more surgical helmets, which the disinfection device comprises
at least one box-shaped disinfection chamber, presenting a plurality of side walls defining an internal compartment configured to accommodate within it a single helmet to be disinfected and having a prevalent extension along a sagittal plane of the helmet;
means for supplying a disinfectant substance, in communication with the interior of said disinfection chamber;
means of printing configured to generate a label that can be applied to the helmet and containing information about the disinfection.

2. The disinfection device according to claim 1, comprising a pair of disinfection chambers arranged juxtaposed one to the other, each box-shaped, having a prevalent extension along a sagittal plane and configured to house a respective helmet to be disinfected, said supplying means being in independent communication with the interior of each of said disinfection chambers.

3. The disinfection device according to claim 1, wherein said or each of said chambers is openable for the introduction of said surgical helmet inside thereof.

4. The disinfection device according to claim 1, wherein said or each chamber has transparent peripheral walls or a transparent viewing window.

5. The disinfection device according to claim 1, comprising means for retaining a helmet, arranged within said or each chamber and preferably of the latching type.

6. The disinfection device according to claim 1, wherein said means for supplying a disinfectant substance are configured for introducing a controlled quantity, or volume, of disinfectant into one or each disinfection chamber.

7. The disinfection device according to claim 1, wherein said means for supplying are configured for a nebulization of a disinfectant substance within said or each chamber.

8. The disinfection device according to claim 1, wherein said printing means are configured to print onto the label one or more information selected from: date, time, parameters of the disinfection process and/or of the substance or substances used.

9. The disinfection device according to claim 1, comprising a support base carrying a user interface.

10. The disinfection device according to claim 1, comprising a control unit of said means for supplying and said means for printing, which control unit is programmed to perform one or more disinfection processes.
